(19) United States
(12) Patent Application Publication
(10) Pub. No.: US 2015/0132322 A1
(43) Pub. Date: May 14, 2015

(54) METHOD OF GENERATING HUMAN MONOCLONAL ANTIBODIES

(71) Applicant: Immune Pharmaceuticals Ltd., Herzliya-Pituach (IL)

(72) Inventors: Jean Elie Kadouche, Paris (FR); Alain Razafindrastita, Anthony (FR)

(21) Appl. No.: 14/399,167

(22) PCT Filed: May 6, 2013
(86) PCT No.: PCT/IL2013/050384

§ 371 Date: Nov. 6, 2014

Related U.S. Application Data
(60) Provisional application No. 61/643,368, filed on May 7, 2012.

(51) Int. Cl. C07K 16/28; A61K 38/19; A61K 35/28; A61K 39/395

(52) U.S. Cl. CPC C07K 16/2884; A61K 39/3955; A61K 38/191; A61K 35/28; A61K 2035/124
USPC 424/174.1; 424/85.1; 530/388.15; 800/8; 435/335; 435/70.21; 435/452

(57) ABSTRACT

This invention provides a human antibody, a hybridoma cell line for the production of the antibody, a reconstituted mouse strain for the production of the hybridoma, and methods of producing and using thereof.

### FIELD OF INVENTION

This invention is directed to a human antibody, a hybridoma cell line for the production of the antibody, a reconstituted mouse strain for the production of the hybridoma, and methods of producing and using thereof.

### BACKGROUND OF THE INVENTION

Antibodies that recognize and adhere to proteins on the surface of bacteria, virus or parasites help immune system cells identify, attack and remove them from the body. Similarly, monoclonal antibodies that adhere to cancer cells but not to normal cells can be an effective therapy for human cancers.

Hybridomas are hybrid cell lines that are used to reliably produce monoclonal antibodies. Hybridomas are made by fusing a specific antibody-producing B cell with a myeloma cell that is selected for its ability to grow in tissue culture and for an absence of antibody chain synthesis. The B cell is obtained from lymphocytes obtained from an animal, usually a mouse, that has been immunized with an antigen of interest. After immunization, lymphocytes are isolated and then fused with an immortal myeloma cell line using a suitable fusing agent. Myeloma cells that fuse efficiently, support stable high-level production of antibody by the selected antibody-producing cells, and are sensitive to a medium that selects against the unfused parental cells are sought as fusion partners.

Mouse-human hybrid myeloma (heteromyeloma) cell lines have been constructed. These heteromyelomas include: a) Mouse myeloma X63-Ag8.653 fused with human myeloma U-266; b) HAB-1 derived from hybrization of murine Ag8 myeloma cells and lymph node cells from a patient with a b-cell lymphoma; and c) NS1 mouse myeloma cell line fused with human lymphocytes. Another heteromyeloma, K6H6/B5 (ATCC CRL1823), was derived from a fusion of murine NS1-Ag4 myeloma cells with cells from a human nodular lymphoma. However, the yield of viable hybrids with these heteromyelomas was too low, probably because of their slow growth.

One strategy for effectively producing human antibodies in mice has been using immunodeficient mice for reconstituting human hematopoiesis. Human fetal tissues, including fetal liver hematopoietic cells, thymus, and lymph nodes, have been transplanted into SCID mice to induce mature human T- and B-cell development. Transferring human blood mononuclear cells into SCID mice has been used to reconstitute the immune system of mice to produce human T and B cells. These initial studies suggested the usefulness of immunodeficient mice for reconstitution of the human lymphoid system from human bone marrow hematopoietic stem cells (HSCs). A number of other studies have since aimed at reconstituting human immunity. In addition to SCID mice, other mutants such as Rag1^{-/-} or Rag2^{-/-} have been used. However, the levels of engraftment in these models were still low, presumably due to the remaining innate immunity of host animals. Moreover, these models present the strong disadvantage that the quantitative T cell reconstitution is very poor, with a very limited number of T cells in the chimera lymphoid organs.

In the field of antibody-based therapy, the use of chimeric (human-mouse) and humanized antibodies for prevention and treatment of diseases in human patients has been attempted. However, these antibodies can be rejected by the host's immune system, resulting in potentially life-threatening side-effects. Therefore to circumvent these obstacles, it would be highly advantageous to produce and make use of fully human antibodies for prophylactic and therapeutic purposes against diseases such as cancer and infectious diseases. The present invention addresses this need by providing a method to obtain immunodeficient mice reconstituted with human hematopoietic cells and tissues. The reconstituted mice are thus capable of producing fully human antibodies specific for an antigen, i.e. antibodies wherein both the variable and the constant regions are of human origin. Splenocytes from these mice may be used as a source of lymphocytes for creating human hybridomas of the present invention.

### SUMMARY OF THE INVENTION

In one embodiment, the subject invention provides a method for obtaining a reconstituted mouse capable of producing a fully human antibody, the method comprising the steps of: (a) administering a neutralizing antibody specific for murine IL-2R beta to an immunodeficient mouse; (b) administering human hematopoietic stem cells to the immunodeficient mouse; and (c) administering human TNF-α to the mouse, thereby obtaining a mouse capable of producing a fully human antibody.

In another embodiment, the subject invention provides a kit for obtaining a reconstituted immunodeficient mouse capable of producing a fully human antibody, the kit comprising: (a) neutralizing antibody specific for murine IL-2R beta; (b) human hematopoietic stem cells; (c) human TNF-α; and (d) instructions for use thereof.

In another embodiment, the subject invention provides an immunologically reconstituted mouse capable of producing a fully human antibody made according to the method comprising the steps of: (a) administering a neutralizing antibody specific for murine IL-2R beta to an immunodeficient mouse; (b) administering human hematopoietic stem cells to the immunodeficient mouse; and (c) administering human TNF-α to the mouse, thereby obtaining a mouse capable of producing a fully human antibody.

In another embodiment, the subject invention provides a method of isolating a human antibody targeting an antigen of interest, the method comprising the steps of: (a) administering an antigen of interest to the reconstituted mouse capable of producing a fully human antibody as described herein; (b) obtaining a splenocyte that produces human antibodies specific for said antigen from said mouse; (c) fusing said splenocyte to a heteromyeloma to produce a hybridoma cell capable of producing a human antibody specific for said antigen; and (d) isolating a human antibody from said hybridoma cell that is specific for said antigen, thereby isolating a human antibody.

In another embodiment, the subject invention provides a humanized antibody isolated using a method comprising the steps of: (a) administering an antigen of interest to the reconstituted mouse capable of producing a fully human antibody as described herein; (b) obtaining a splenocyte that produces human antibodies specific for said antigen from said mouse; (c) fusing said splenocyte to a heteromyeloma to produce a hybridoma cell capable of producing a human antibody specific for said antigen; and (d) isolating a human antibody from said hybridoma cell that is specific for said antigen, thereby isolating a human antibody.

In another embodiment, the subject invention provides a method of treating cancer, the method comprising the step of administering to a subject a humanized antibody isolated using a method comprising the steps of: (a) administering an antigen of interest to the reconstituted mouse capable of producing a fully human antibody as described herein; (b) obtaining a splenocyte that produces human antibodies specific for said antigen from said mouse; (c) fusing said splenocyte to a heteromyeloma to produce a hybridoma cell capable of producing a human antibody specific for said antigen; and (d) isolating a human antibody from said hybridoma cell that is specific for said antigen, thereby isolating a human antibody.

In another embodiment, the subject invention provides a method of preventing, inhibiting, or suppressing cancer, the method comprising the step of administering to a subject a humanized antibody isolated using a method comprising the steps of: (a) administering an antigen of interest to the reconstituted mouse capable of producing a fully human antibody as described herein; (b) obtaining a splenocyte that produces human antibodies specific for said antigen from said mouse; (c) fusing said splenocyte to a heteromyeloma to produce a hybridoma cell capable of producing a human antibody specific for said antigen; and (d) isolating a human antibody from said hybridoma cell that is specific for said antigen, thereby isolating a human antibody.

In another embodiment, the subject invention provides a method of producing a hybridoma cell line capable of secreting a human antibody specific to an antigen of interest, the method comprising the steps of: (a) administering an antigen of interest to the reconstituted mouse capable of producing a humanized antibody as described herein; (b) obtaining a splenocyte that produces human antibodies specific for said antigen from said mouse; (c) fusing said splenocyte with a heteromyeloma to form a hybridoma cell capable of producing a human antibody specific for said antigen; (d) identifying a hybridoma cell specific for said antigen; and (e) expanding said hybridoma cell to produce a hybridoma cell culture capable of producing a human antibody specific for said antigen, thereby producing a hybridoma cell line capable of secreting a human antibody specific to an antigen of interest.

In another embodiment, the subject invention provides a hybridoma cell line produced by the process comprising the steps of: (a) administering an antigen of interest to the reconstituted mouse capable of producing a humanized antibody as described herein; (b) obtaining a splenocyte that produces human antibodies specific for said antigen from said mouse; (c) fusing said splenocyte with a heteromyeloma to form a hybridoma cell capable of producing a human antibody specific for said antigen; (d) identifying a hybridoma cell specific for said antigen; and (e) expanding said hybridoma cell to produce a hybridoma cell culture capable of producing a human antibody specific for said antigen, thereby producing a hybridoma cell line capable of secreting a human antibody specific to an antigen of interest.

In another embodiment, the subject invention provides a kit for preparing a hybridoma cell line capable of secreting a human antibody specific to an antigen of interest, the kit comprising: (a) a splenocyte producing human antibodies specific for the antigen from a reconstituted immunodeficient mouse administered a neutralizing antibody specific for murine IL-2R beta; human hematopoietic stem cells; human TNF-α; and the antigen of interest; (b) a heteromyeloma for fusing with the splenocyte; and (c) instructions for use thereof.

In another embodiment, the subject invention provides a heteromyeloma cell produced by the fusion of the modified human lymphoma B cell line (OCI-LY-19, DSMZ no ACC 528) to the murine myeloma X63-Ag8.653 (ATCC CRL1580).

In another embodiment, the subject invention provides a method of producing a heteromyeloma cell comprising the step of fusing the modified human lymphoma B cell line (OCI-LY-19, DSMZ no ACC 528) to the murine myeloma X63-Ag8.653 (ATCC CRL1580), thereby producing a heteromyeloma.

In another embodiment, the subject invention provides a kit for preparing a heteromyeloma cell line, the kit comprising: (a) murine myeloma X63-Ag8.653 cells; (b) modified human B lymphoma OCI-LY-19, DSMZ no ACC 528 cells; and (c) instructions for use thereof.

In another embodiment, the subject invention provides a method of isolating a human antibody specific for an antigen of interest, the method comprising the steps of: (a) administering an antigen of interest to a mouse capable of producing a fully human antibody; (b) obtaining a splenocyte that produces human antibodies specific for said antigen from said mouse; (c) fusing said splenocyte to the heteromyeloma as described herein to form a hybridoma cell capable of producing a human antibody specific for said antigen; and (d) isolating a human antibody from said hybridoma cell that is specific for said antigen, thereby isolating a human antibody.

In another embodiment, the subject invention provides a humanized antibody isolated according to the method comprising the steps of: (a) administering an antigen of interest to a mouse capable of producing a fully human antibody; (b) obtaining a splenocyte that produces human antibodies specific for said antigen from said mouse; (c) fusing said splenocyte to the heteromyeloma as described herein to form a hybridoma cell capable of producing a human antibody specific for said antigen; and (d) isolating a human antibody from said hybridoma cell that is specific for said antigen, thereby isolating a human antibody.

In another embodiment, the subject invention provides a method of treating cancer, the method comprising the step of administering to a subject a humanized antibody isolated according to the method comprising the steps of: (a) administering an antigen of interest to a mouse capable of producing a fully human antibody; (b) obtaining a splenocyte that produces human antibodies specific for said antigen from said mouse; (c) fusing said splenocyte to the heteromyeloma as described herein to form a hybridoma cell capable of producing a human antibody specific for said antigen; and (d) isolating a human antibody from said hybridoma cell that is specific for said antigen, thereby isolating a human antibody.

In another embodiment, the subject invention provides a method of preventing, inhibiting, or suppressing cancer, the method comprising the step of administering to a subject a humanized antibody isolated according to the method comprising the steps of: (a) administering an antigen of interest to a mouse capable of producing a fully human antibody; (b) obtaining a splenocyte that produces human antibodies specific for said antigen from said mouse; (c) fusing said splenocyte to the heteromyeloma as described herein to form a hybridoma cell capable of producing a human antibody specific for said antigen; and (d) isolating a human antibody from said hybridoma cell that is specific for said antigen, thereby isolating a human antibody.

In another embodiment, the subject invention provides a method of producing a hybridoma cell line capable of secreting a human antibody specific to an antigen of interest, the method comprising the steps of: (a) administering an antigen of interest to a mouse capable of producing a fully human antibody; (b) obtaining a splenocyte that produces human antibodies specific for said antigen from said mouse; (c) fusing said splenocyte to the heteromyeloma as described herein to form a hybridoma capable of producing a human antibody specific for said antigen; (d) identifying a hybridoma cell specific for said antigen; and (e) expanding said hybridoma cell to produce a hybridoma cell culture capable of producing a human antibody specific for said antigen, thereby producing a hybridoma cell line capable of secreting a human antibody specific to an antigen of interest.

In another embodiment, the subject invention provides a hybridoma produced by the method comprising the steps of: (a) administering an antigen of interest to a mouse capable of producing a fully human antibody; (b) obtaining a splenocyte that produces human antibodies specific for said antigen from said mouse; (c) fusing said splenocyte to the heteromyeloma as described herein to form a hybridoma capable of producing a human antibody specific for said antigen; (d) identifying a hybridoma cell specific for said antigen; and (e) expanding said hybridoma cell to produce a hybridoma cell culture capable of producing a human antibody specific for said antigen, thereby producing a hybridoma cell line capable of secreting a human antibody specific to an antigen of interest.

In one embodiment, the invention relates to a kit for preparing a hybridoma cell line capable of secreting a human antibody specific to an antigen of interest, the kit comprising: (a) a splenocyte that produces human antibodies specific for the antigen from a mouse capable of producing a fully human antibody; (b) a heteromyeloma cell produced by the fusion of the modified human lymphoma B cell line (OCI-LY-19, DSMZ no ACC 528) to the murine myeloma X63-Ag8.653 (ATCC CRL1580) for fusing with the splenocyte; and (c) instructions for use thereof.

Other features and advantages of the present invention will become apparent from the following detailed description examples and figures. It should be understood, however, that the detailed description and the specific examples while indicating preferred embodiments of the invention are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present disclosure, the inventions of which can be better understood by reference to one or more of these drawings in combination with the detailed description of specific embodiments presented herein. The patent or application file contains at least one drawing executed in color. Copies of this patent or patent application publication with color drawing(s) will be provided by the Office upon request and payment of the necessary fee.

FIG. 1 shows human IgM and IgG ELISA. Levels of human IgM and IgG were measured in humanized mice sera by an ELISA assay with anti-human IgG or anti-human IgM (Jackson) for coating and biotinylated mouse anti-human IgG for detection.

FIG. 2 shows FACS analysis of spleen from humanized mice. Spleens were digested, stained with anti-human CD45, CD20 and CD3 antibodies and analyzed by FACS. Samples were gated on live lymphocytes by forward and side scatter and on CD45 positive cells.

FIG. 3 shows immunohistochemistry of spleen from humanized mice. Spleens were fixed in neutral buffered formalin, embedded in paraffin, and then 5 μm tissue sections were cut. Staining was performed with anti-human CD45 and anti-CD20 Mabs.

FIG. 4 shows the production of fully human antibodies. Splenocytes and mouse heteromyeloma K6H6/B5 (ATCC) were fused according to the method of Kohler and Milstein. After cloning, stable positive clones (18 IgM, 5 IgG) producing specific antibodies against the antigen were obtained. ELISA tests were performed by coating the antigen.

FIG. 5 shows a survival analysis after treatment with IMP111 in a human AML1 mouse model. A therapeutic effect is seen in the IMP111 treated group as opposed to the IgG1 control.

FIG. 6 shows antibody-dependent cellular phagocytosis (ADCP) of tumor cells.

FIG. 7. Peripheral blood FACS analysis of human AML1 CD44+ cells after treatment with IMP111 (FIG. 7B) in human AML1 mouse model at Day 53. Results show a decrease of CD44+ tumor cells in IMP11 treated groups (FIG. 7B) as opposed to the IgG1-treated control group (FIG. 7A).

FIG. 8 shows a blood smear May-Grünwal-Giemsa (MGG) stain after control (IgG1) treatment (FIG. 8A) and after IMP111 treatment (FIG. 8B) at day 53. Treated group showed no presence of tumor cells, whereas control groups showed massive hematopoietic tumor invasion by blast cells type.

FIG. 9 shows an image of two spleens, one from the IMP111 treated group of mice (smaller spleen) and the other from the control treated mice (larger spleen).

### DETAILED DESCRIPTION OF THE INVENTION

It has been the norm to use chimeric (human-mouse) and humanized antibodies for prevention and treatment of diseases in human patients. However, these antibodies have shortcomings that range from rejection of the antibody by the patient's immune system to the occurrence of antibody-induced and potentially life-threatening, side effects. As such, it would be highly advantageous to make use of fully human antibodies for disease prophylaxis and therapy in human patients, as doing so should obviate these shortcomings with minimal to no side-effects while retaining maximum efficacy.

The present invention provides reconstituted mice capable of producing fully human antibodies and capable of maintaining immunity for long periods. The invention further provides a method of preparing human antibodies from these mice. More specifically, the present invention provides reconstituted mice having a human immune system that is constructed by transplanting human hematopoietic stem cells (HSC) such as CD34⁺ cells into NOD SCID mice. The invention further provides methods for preparing human antibodies using the reconstituted mice, and human antibodies prepared by these methods.

Hence, in one embodiment, the invention provides a method for obtaining a reconstituted mouse capable of producing a fully human antibody, the method comprising the steps of: (a) administering a neutralizing antibody specific for murine IL-2R beta to an immunodeficient mouse; (b) administering human hematopoietic stem cells to the immunodeficient mouse; and (c) administering human TNF-α to the mouse, thereby obtaining a mouse capable of producing a fully human antibody.

In one embodiment, the subject invention provides a method for obtaining a reconstituted mouse capable of producing a fully human antibody, the method comprising the steps of: (a) administering a neutralizing antibody specific for murine IL-2R beta to an immunodeficient mouse; (b) administering human hematopoietic stem cells to the immunodeficient mouse; and (c) administering human TNF-α to the mouse, thereby obtaining a mouse capable of producing a fully human antibody.

In another embodiment, the subject invention provides a kit for obtaining a reconstituted immunodeficient mouse capable of producing a fully human antibody, the kit comprising: (a) neutralizing antibody specific for murine IL-2R beta; (b) human hematopoietic stem cells; (c) human TNF-α; and (d) instructions for use thereof.

In another embodiment, the subject invention provides an immunologically reconstituted mouse capable of producing a fully human antibody made according to the method comprising the steps of: (a) administering a neutralizing antibody specific for murine IL-2R beta to an immunodeficient mouse; (b) administering human hematopoietic stem cells to the immunodeficient mouse; and (c) administering human TNF-α to the mouse, thereby obtaining a mouse capable of producing a fully human antibody.

The term “human antibody”, in one embodiment, is intended to include antibodies having variable regions in which both the framework and CDR regions are derived from human germline immunoglobulin sequences. Furthermore, if the antibody contains a constant region, the constant region also is derived from human germline immunoglobulin sequences. The human antibodies of the invention may include amino acid residues not encoded by human germline immunoglobulin sequences (e.g., mutations introduced by random or site-specific mutagenesis in vitro or by somatic mutation in vivo). However, the term “human antibody”, in another embodiment, is not intended to include antibodies in which CDR sequences derived from the germline of another mammalian species, such as a mouse, have been grafted onto human framework sequences.

In one embodiment, the term “human monoclonal antibody” refers to antibodies displaying a single binding specificity which have variable regions in which both the framework and CDR regions are derived from human germline immunoglobulin sequences. In one embodiment, the human monoclonal antibodies are produced by a hybridoma which includes a splenocyte, more specifically, a B cell obtained from a transgenic nonhuman animal, e.g., a transgenic mouse, having a genome comprising a human heavy chain transgene and a light chain transgene, fused to an immortalized cell such as a myeloma or heteromyeloma.

In one embodiment, the term “antibody” refers to monoclonal antibodies (including full length monoclonal antibodies) of any isotype such as IgG, IgM, IgA, IgD, and IgE, polyclonal antibodies, multispecific antibodies, chimeric antibodies, and antibody fragments. An antibody reactive with a specific antigen can be generated by immunizing an animal with the antigen or an antigen-encoding nucleic acid.

Monoclonal antibodies (mAbs) of the present invention can be produced by a variety of techniques, including conventional monoclonal antibody methodology e.g., the standard somatic cell hybridization technique of Kohler and Milstein (1975 Nature 256:495). Although somatic cell hybridization procedures are preferred, in principle, other techniques for producing a monoclonal antibody can be employed including, but not limited to, viral or oncogenic transformation of B lymphocytes.

The preferred animal system for preparing hybridomas is the murine system. Hybridoma production in the mouse is a very well-established procedure. Immunization protocols and techniques for isolation of immunized splenocytes for fusion are known in the art. Fusion partners (e.g., murine myeloma cells) and fusion procedures are also known.

In one embodiment, the phrases “an antibody recognizing an antigen” and “an antibody specific for an antigen” are used interchangeably herein with the term “an antibody which binds specifically to an antigen.”

In another embodiment, the invention provides an immunologically reconstituted mouse for producing a fully human antibody.

In another embodiment, the invention provides a method of isolating a human antibody targeting an antigen of interest, the method comprising the steps of: (a) administering an antigen of interest to the reconstituted mouse capable of producing a fully human antibody provided herein (b) obtaining a splenocyte that produces human antibodies specific for the antigen from the mouse; (c) fusing the splenocyte to a heteromyeloma to produce a hybridoma cell capable of producing a human antibody specific for the antigen; (d) isolating a human antibody from the hybridoma cell that is specific for the antigen; (e) identifying a hybridoma cell from step (c) that is specific for the antigen; (f) expanding the hybridoma cell to produce a hybridoma cell culture capable of producing a human antibody specific for the antigen; and (g) isolating a human antibody which binds to the antigen from one or more cells of the hybridoma cell culture. In another embodiment, the step of administering the antigen to the mouse primes or enables splenocytes in the mouse to produce human antibodies specific for the antigen.

In one embodiment, the term “capable of” refers to the ability the reconstituted mouse has to produce a fully human antibody. In another embodiment, the term carries an implied functional meaning that the mouse can fully produce a human antibody according to the methods and guidance provided herein. In one embodiment, the term refers to a mouse that is able to produce a fully human antibody, whether or not it is being produced at the moment in time. In another embodiment, the reconstituted mouse of the present invention produces a fully human antibody.

In another embodiment, provided herein is a method of producing a hybridoma cell line capable of secreting a human antibody specific to an antigen of interest, the method comprising the steps of: (a) administering an antigen of interest to the reconstituted mouse capable of producing a humanized antibody provided herein; (b) obtaining a splenocyte that produces human antibodies specific for the antigen from the mouse; (c) fusing the splenocyte with a heteromyeloma to form a hybridoma cell capable of producing a human antibody specific for the antigen; (d) identifying a hybridoma cell specific for the antigen; and (e) expanding the hybridoma cell to produce a hybridoma cell culture capable of producing a human antibody specific for the antigen.

In another embodiment, the subject invention provides a method of producing a hybridoma cell line capable of secreting a human antibody specific to an antigen of interest, the method comprising the steps of: (a) administering an antigen of interest to a mouse capable of producing a fully human antibody; (b) obtaining a splenocyte that produces human antibodies specific for said antigen from said mouse; (c) fusing said splenocyte to the heteromyeloma as described herein to form a hybridoma capable of producing a human antibody specific for said antigen; (d) identifying a hybridoma cell specific for said antigen; and (e) expanding said hybridoma cell to produce a hybridoma cell culture capable of producing a human antibody specific for said antigen, thereby producing a hybridoma cell line capable of secreting a human antibody specific to an antigen of interest.

In another embodiment, the subject invention provides a hybridoma produced by the method comprising the steps of: (a) administering an antigen of interest to a mouse capable of producing a fully human antibody; (b) obtaining a splenocyte that produces human antibodies specific for said antigen from said mouse; (c) fusing said splenocyte to the heteromyeloma as described herein to form a hybridoma capable of producing a human antibody specific for said antigen; (d) identifying a hybridoma cell specific for said antigen; and (e) expanding said hybridoma cell to produce a hybridoma cell culture capable of producing a human antibody specific for said antigen, thereby producing a hybridoma cell line capable of secreting a human antibody specific to an antigen of interest.

In one embodiment, the invention relates to a kit for preparing a hybridoma cell line capable of secreting a human antibody specific to an antigen of interest, the kit comprising: (a) a splenocyte that produces human antibodies specific for the antigen from a mouse capable of producing a fully human antibody; (b) a heteromyeloma cell produced by the fusion of the modified human lymphoma B cell line (OCI-LY-19, DSMZ no ACC 528) to the murine myeloma X63-Ag8.653 (ATCC CRL1580) for fusing with the splenocyte; and (c) instructions for use thereof.

In another embodiment, prior to the step of isolating a human antibody, the fused cell line or hybridoma which produces antibodies, is grown briefly in culture and then re-injected into another mouse's peritoneum. Finally, the ascites fluid which contains monoclonal antibodies is harvested from the mouse and the monoclonal antibody is isolated from the ascites fluid by methods known in the art (see for example, Ball W. J. et al. The Journal of Immunology, 1999, 163: 2291-2298).

In one embodiment, the binding specificity of monoclonal antibodies produced by hybridoma cells is determined by immunoprecipitation or by an in vitro binding assay, such as radioimmunoassay (RIA) or enzyme linked immuno-absorbent assay (ELISA). The binding affinity of the monoclonal antibody can, for example, be determined by the Scatchard analysis of Munson et al., 1980, Anal. Biochem., 107:220. After hybridoma cells are identified that produce antibodies of the desired specificity, affinity, and/or activity, the clones may be subcloned by limiting dilution procedures and grown by standard methods (Goding, Monoclonal Antibodies: Principles and Practice, pp. 59 103 (Academic Press, 1986)). Suitable culture media for this purpose include, for example, D-MEM or RPMI 1640 medium. In addition, the hybridoma cells may be grown in vivo as ascites tumors in an animal. The monoclonal antibodies secreted by the subclones are suitably separated from the culture medium, ascites fluid, or serum by conventional immunoglobulin purification procedures such as, for example, protein A Sepharose, hydroxylapatite chromatography, gel electrophoresis, dialysis, or affinity chromatography.

Human monoclonal antibodies of the invention can also be prepared using phage display methods for screening libraries of human immunoglobulin genes. Such phage display methods for isolating human antibodies are established in the art. See for example: U.S. Pat. Nos. 5,223,409; 5,403,484; and 5,571,698 to Ladner et al.; U.S. Pat. Nos. 5,427,908 and 5,580,717 to Dower et al.; U.S. Pat. Nos. 5,969,108 and 6,172,197 to McCafferty et al.; and U.S. Pat. Nos. 5,885,793; 6,521,404; 6,544,731; 6,555,313; 6,582,915, and 6,593,081 to Griffiths et al.

In one embodiment, binding of a human antibody provided herein to its target is determined using a binding assay. Exemplary binding assays include Fluorescent Activated Cell Sorting (FACS) and ELISA-based binding assays. In another embodiment, affinity of a human antibody provided herein to its target antigen is determined using an affinity assay, including, but not limited to, an ELISA-based affinity assay, Surface Plasmon Resonance, and any other method available in the art know to be used for the same purposes.

In one embodiment, the human hematopoietic stem cells reconstitute human B and T cell function in the mouse, which in one embodiment, is an immunodeficient mouse.

In one embodiment, hematopoietic stem cells are are multipotent stem cells that give rise to all the blood cell types from the myeloid (monocytes and macrophages, neutrophils, basophils, eosinophils, erythrocytes, megakaryocytes/platelets, dendritic cells), and lymphoid lineages (T-cells, B-cells, NK-cells). These cells can be obtained from bone marrow, peripheral blood, umbilical cord blood, embryonic stem cells.

In one embodiment, the hematopoietic stem cell is an umbilical cord blood cell. In another embodiment, the umbilical cord blood cell is a CD34⁺ cell. In another embodiment, the CD34⁺ cell is a human CD34⁺ cell. In one embodiment, human CD34⁺ cells originate from human umbilical cord blood. In another embodiment, the human CD34⁺ cells originate from human bone marrow. In one embodiment, the CD34+ cell is grown in culture media suitable for growth and division of the cell without affecting its stem cell properties. In another embodiment, a suitable amount of Notch1 necessary to preserve the stem cell properties is used to grow the CD34+ cell. In another embodiment, Notch1 stimulates the cells to divide without sacrificing their key stem cell properties.

In one embodiment, the term “tumor necrosis alpha” or “TNF-α”, refers to a cytokine involved in the regulation of immune cells. TNF-α according to the present invention is preferentially of human origin. Human TNF-α is a non-glycosylated protein of 17 kDa and a length of 157 amino acids. More preferentially, human TNF-α has an amino acid sequence represented by NP_{—}000585. TNF-alpha enhances the proliferation of T-cells induced by various stimuli in the absence of IL-2. Administration of human TNF-α thus favours the generation of functional human T cells from the CD34⁺ cells.

In another embodiment, the TNF-α enables the generation of functional human T cells from the hematopoietic stem cells. In another embodiment, the TNF-α skews differentiation of hematopoietic stem cells towards a T cell phenotype. In another embodiment, the TNF-α allows the differentiation of hematopoietic stem cells into functional T cells.

In one embodiment, the method provided herein further comprises the step of administering to mice an antibody specific for an antigen prior to administering the antigen. In another embodiment, administering the antibody to the mouse prior to administering the antigen results in an enhanced secondary immune response to the antigen in the mouse. In another embodiment, the method is optimized for proliferation of secondary cell-dependent immune response (T cell) rather than a primary, innate immune response (in one embodiment, a B cell immune response, in another embodiment, a T cell-independent immune response).

In one embodiment, the immunodeficient mouse provided herein is a NOD_SCID mouse. According to the method of the invention, human CD34⁺ cells are transplanted into pre-conditioned immunodeficient recipient mice. While the invention can be performed with any mouse whose immune system is already compromised, it is preferable to use a SCID mouse. NOD SCID mice have an impaired T- and B-cell lymphocyte function and lack NK function and the ability to stimulate complement activity. Such NOD SCID mice are known in the art (J. Immunol., 154: 180, 1995). In one embodiment, the NOD_SCID mouse is NOD.CB 17-Prkdcscid/SzJ, in another embodiment, the NOD_SCID mouse is NOD.129S7 (B6)-Rag1tm1Mom/J.

In one embodiment, the term “CD34” refers to a human cell surface glycoprotein having an amino acid sequence as in Genbank Accession No. NP_{—}598415 and expressed selectively on human hematopoietic progenitor cells. Thus, the term “human CD34⁺ cells” herein refers to a population of human cells carrying CD34 as a cell surface antigen, the population of hematopoietic stem cells. The source of CD34⁺ cells is not limited, but those prepared from human umbilical cord blood are preferably used. In this case, the human CD34⁺ cells used in the method of the invention can be prepared extemporaneously, i.e. the CD34⁺ cells transplanted into the host mouse are those immediately separated from the human umbilical cord blood. It is to be understood that a skilled artisan can employ the use of methods available in the art for enriching and/or preparing human CD34+ cells, including density centrifugation, affinity or cell enrichment columns, filter-based enrichment, magnetic beads, cell sorting, and the like.

Alternatively, the human CD34+ cells can be cultivated and stored by freezing until needed. The culture may be performed using an irradiated mouse bone marrow stromal cell line (such as HESS-5 cells) as a feeder cell, with human SCF, human TPO, and human FL (Flk-2/Flt-3 ligand) added. The molecules are preferably added at around 50 ng/ml. A protocol for preparing human CD34⁺ is described in Example 1. In the method for transplanting human CD34⁺ cells or accessory cells into mice, there is no limitation on the route of transplantation so long as the method enables the transfer of those cells into the blood stream. However, intravenous injection, and in particular injection through the tail veil, is preferably used because it is easily manipulated. In one embodiment, the number of CD34⁺ cells transplanted is comprised between 1× and 10×10⁶ cells; more preferably, between 2× and 5×10⁶ cells; even more preferably, between 3×10⁶ and 4×10⁶ cells.

The CD34⁺ cells of the invention can be used for transplantation into a standard SCID mouse. However, if such a mouse is used, NK cell-based cytotoxicity against the transplanted CD34⁺ cells may cause a lowering of the engraftment ratio of the transplanted cells. It is thus desirable according to the invention to use NOD SCID mice, i.e. SCID mice whose NK cells have reduced activity. Moreover, treating the NOD SICD mice with an efficient amount of an antibody directed against murine IL-2R beta substantially wipes out all NK cell-cytotoxic activity.

In one embodiment, provided herein is an anti-IL-2R-antibody that eliminates natural killer (NK) cell-cytotoxic activity. In another embodiment, the antibody suppresses NK cell-cytotoxic activity.

By “interleukin 2 receptor” or “IL-2R”, it is herein referred to the cellular receptor for the interleukin-2 (IL-2) cytokine. By “interleukin-2”, or “IL-2”, it is herein referred to a cytokine with immunoregulatory properties. In one embodiment, IL-2 according to the invention has an amino acid sequence represented by NP_{—}032392 (mouse) or NP_{—}000577 (human). IL-2 is required, amongst others, for 1) enhancement of lymphocyte mitogenesis and stimulation of long-term growth of IL-2-dependent cell lines; 2) enhancement of lymphocyte cytotoxicity; 3) induction of killer cell (lymphokine-activated (LAK) and natural (NK)) activity; and 4) induction of interferon-gamma production.

The IL-2R receptor is involved in T cell-mediated immune responses and is present in 3 forms with respect to its ability to bind interleukin 2. The low affinity form is a monomer of the alpha subunit and is not involved in signal transduction. The intermediate affinity form consists of an alpha/beta subunit heterodimer, while the high affinity form consists of an alpha/beta/gamma subunit heterotrimer. Both the intermediate and high affinity forms of the receptor are involved in receptor-mediated endocytosis and transduction of mitogenic signals from interleukin 2.

In one embodiment, treating the NOD SCID mice with an efficient amount of an antibody directed against murine IL-2R beta is more efficient and cost-effective than generating an IL-2R knockout mouse. Whereas the deletion of the gene encoding the gamma subunit leads to the disappearance of only the high affinity form, leaving the intermediate form intact, administration of a neutralizing antibody directed to murine IL-2R beta leads to functional inactivation of both the intermediate and high affinity forms (Ito et al., Blood, 100(9): 3175-3182, 2002; Ishikawa et al., Blood, 106(5): 1565-1573). The administration of a neutralizing antibody directed to murine IL-2R beta according to the invention thus presents the advantage of eliminating all signal transduction from IL-2. Hence, NK cells are completely depleted by this treatment, thus improving the engraftment capacity of the NOD SCID mice known in the art.

In addition, it is much easier and quicker to obtain NK depletion by administration of an antibody neutralizing IL-2R beta than by introducing a homozygous deletion of the IL-2R gamma subunit gene. For example, there is no need to perform a tedious deletion of one allele after the other, no need to identify the mouse carrying the correct genotype, no need to backcross the resulting transgenic mouse in the desired genetic background. All that is required is the administration of an antibody.

In one embodiment, the terminology “neutralizing antibody directed to murine IL-2R beta”, refers to an antibody which is capable of binding to the murine IL-2R beta protein and preventing any IL2-R-mediated signaling. In another embodiment, the “murine IL-2R beta” according to the invention is a polypeptide having the amino acid sequence represented by NP_{—}032394. Antibodies recognizing murine IL-2R beta have been previously described (Francois et al., J. Immunol., 150(10): 4610-4619, 1993; Tournoy et al., Eur. J. Immunol., 28(10): 3221-3230; Schultz et al., Exp. Hematol., 31: 551-558, 2003). Such antibodies are also commercially available (e.g. Santa Cruz: sc-52571, sc-1044, or sc-80081).

Optionally, the NOD SCID mouse of the invention is irradiated prior to the administration of IL-2R beta antibody. It is to be understood that a specific irradiation dose required to achieve the desired effect (e.g. murine immune-incapacitation) can be empirically determined by a skilled artisan. In another embodiment, the mice provided herein are irradiated using 3.25 Gy or 325 rads, where 100 rads=1 gray (Gy). In another embodiment, the mice provided herein are irradiated using 100-200 rads. In another embodiment, the mice provided herein are irradiated using 201-300 rads. In another embodiment, the mice provided herein are irradiated using 301-400 rads. In another embodiment, the mice provided herein are irradiated using 501-600 rads. In another embodiment, the mice provided herein are irradiated using 701-800 rads. In another embodiment, the mice provided herein are irradiated using 801-900 rads. In another embodiment, the mice provided herein are irradiated using 901-1000 rads. In another embodiment, the mice provided herein are irradiated using 1001-2000 rads. In another embodiment, the mice provided herein are irradiated using 2001-3000 rads. In another embodiment, the mice provided herein are irradiated using 3001-4000 rads. In another embodiment, the mice provided herein are irradiated using 4001-5000 rads. In another embodiment, the mice provided herein are irradiated using 5001-10000 rads. It is to be understood by a skilled artisan that the radiation dose is decreased or increased to achieve the desired effect. In another embodiment, the mice are sub-lethally irradiated. In another embodiment, the mice are lethally irradiated. In another embodiment, the mice provided herein are irradiated until their immune system is incapacitated. Each possibility is considered a separate embodiment of the invention.

In one embodiment, the irradiation of mice provided herein is carried out according to know methods in the art which include but are not limited to, gamma irradiation, X-ray irradiation, UVB-radiation, or a combination thereof.

In one embodiment, the anti-IL-2R antibody provided herein is an anti-mouse CD122.

Since the mouse has mature B cells and T cells differentiated from human immature cells, it is possible using the reconstituted mouse of the invention to prepare an antibody against any antigen, including an antigen of human origin. In one embodiment, expansion of B cell numbers following immunization and boosts enhances the immune response to antigens that produce low titers of antibodies in mammals. In another embodiment in rodents, this method of the invention is useful in the generation of antigen-specific IgG mAbs. In fact, it is possible to efficiently produce an IgG antibody, inducing the antibody class switch by stimulating the reconstituted mouse or the immunocompetent cells, such as spleen cells from the reconstituted mouse, with a B cell expansion agent.

In this embodiment of the invention, the reconstituted NOD SCID mouse of the invention is immunized with an antigen by techniques well known to those skilled in the art. The antigen can be a protein or nucleic acid and a T cell dependent antigen or a T cell independent antigen (including lipids and carbohydrates). T cell independent antigens include bacterial polysaccharides, polymeric proteins and lipopolysaccharides (LPS) and can directly stimulate naive B cells to produce strong antibody responses (generally IgM) in the absence of direct T cell helper functions.

Therefore, the invention also provides a method of producing a human antibody, comprising the steps of: (a) immunizing with an antigen the reconstituted mouse of the invention, and (b) recovering a human antibody which binds to the antigen and which is produced by the immunizing of step (a). Before necessary boosts, a B cell expansion agent is administered to the mouse to generate a higher frequency of antigen-specific B cell clones in Th2-biased hosts.

In one embodiment, the method for obtaining a reconstituted mouse capable of producing a fully human antibody further comprises the step of administering a B cell expansion agent to the immunodeficient mouse. In another embodiment, the method of producing human antibodies may comprise an additional step of administering B cell expansion agent to the reconstituted mouse of the invention. In another embodiment, the B cell expansion agent is BlyS. In another embodiment, the expansion agent induces antibody class switching in the mouse.

In one embodiment, “BlyS” or “B Lymphocyte Stimulator” or “BAFF” or “B-cell activating factor” or “TNF- and APOL-related leukocyte expressed ligand” or “TALL-1” or “Dendritic cell-derived TNF-like molecule” or “CD257” or “tumor necrosis factor ligand superfamily member 13B” refers to a human transmembrane protein of 285 amino acids which in one embodiment, has a sequence as in NP_{—}006564, and derivatives thereof. In another embodiment, BlyS refers to the 152 amino acid-long soluble form of the protein. This form stimulates B lymphocytes to undergo proliferation and to counter apoptosis.

Antibodies generated using a B cell expansion agent or fragments thereof can be raised against an appropriate immunogenic antigen and/or a portion thereof (including synthetic molecules, such as synthetic peptides). Other specific or general antibodies, including, without limitation, mammalian antibodies, can be similarly raised. Preparation of immunogenic antigens and monoclonal antibody production can be performed using any suitable technique.

In one approach, a hybridoma is produced by fusing a suitable immortal cell line with antibody producing cells. In one embodiment, an immortal cell line is a myeloma cell line, which in one embodiment, is Sp2/0, Sp2/0-AG14, NSO, NS1, NS2, AE-I, L.5, L243, 63Ag8.653, Sp2 SA3, Sp2 MAI, Sp2 SS1, Sp2 SA5, U937, MLA 144, ACT IV, MOLT4, DA-I, JURKAT, WEHI, K-562, COS, RAJI, NIH 3T3, HL-60, MLA 144, NAMALWA, NEURO 2A, or the like. In another embodiment, an immortal cell line is a heteromyeloma, fusion product thereof, or any cell or fusion cell derived therefrom, or any other suitable cell line as known in the art (see, e.g., www.atcc.org, www.lifetech.com., and the like). In one embodiment, antibody producing cells, may be, but are not limited to, isolated or cloned spleen, peripheral blood, lymph, tonsil, or other immune or B cell containing cells, or any other cells expressing heavy or light chain constant or variable or framework or CDR sequences, either as endogenous or heterologous nucleic acid, as recombinant or endogenous, viral, bacterial, algal, prokaryotic, amphibian, insect, reptilian, fish, mammalian, rodent, equine, ovine, goat, sheep, primate, eukaryotic, genomic DNA, cDNA, rDNA, mitochondrial DNA or RNA, chloroplast DNA or RNA, hnRNA, mRNA, tRNA, single, double or triple stranded, hybridized, and the like or any combination thereof.

The B cell expansion agent which can be used in the method of the invention may be BLyS, IL-6, APRIL, CD40L, CD154 and anti-IgM/IL4 co-stimulation. The B cell expansion agent used in the present invention may also be a CD40 agonist that increases the number of antigen specific B cells, for example, in the reconstituted mouse being immunized. In one embodiment, the B cell expansion agent is BLyS.

In one embodiment, provided herein is a method of producing a hybridoma cell line capable of producing an antibody with a desired specificity, comprising the steps of: (a) obtaining at least one cell from a reconstituted mouse immunized according to the method of the invention, and (b) fusing the cell with an immortal cell line. Examples of immortal cell lines for use in the invention are further provided herein and known in the art.

In another embodiment, the subject invention provides a method of producing a hybridoma cell line capable of secreting a human antibody specific to an antigen of interest, the method comprising the steps of: (a) administering an antigen of interest to the reconstituted mouse capable of producing a humanized antibody as described herein; (b) obtaining a splenocyte that produces human antibodies specific for said antigen from said mouse; (c) fusing said splenocyte with a heteromyeloma to form a hybridoma cell capable of producing a human antibody specific for said antigen; (d) identifying a hybridoma cell specific for said antigen; and (e) expanding said hybridoma cell to produce a hybridoma cell culture capable of producing a human antibody specific for said antigen, thereby producing a hybridoma cell line capable of secreting a human antibody specific to an antigen of interest.

In another embodiment, the subject invention provides a hybridoma cell line produced by the process comprising the steps of: (a) administering an antigen of interest to the reconstituted mouse capable of producing a humanized antibody as described herein; (b) obtaining a splenocyte that produces human antibodies specific for said antigen from said mouse; (c) fusing said splenocyte with a heteromyeloma to form a hybridoma cell capable of producing a human antibody specific for said antigen; (d) identifying a hybridoma cell specific for said antigen; and (e) expanding said hybridoma cell to produce a hybridoma cell culture capable of producing a human antibody specific for said antigen, thereby producing a hybridoma cell line capable of secreting a human antibody specific to an antigen of interest.

In another embodiment, the subject invention provides a kit for preparing a hybridoma cell line capable of secreting a human antibody specific to an antigen of interest, the kit comprising: (a) a splenocyte producing human antibodies specific for the antigen from a reconstituted immunodeficient mouse administered a neutralizing antibody specific for murine IL-2R beta; human hematopoietic stem cells; human TNF-α; and the antigen of interest; (b) a heteromyeloma for fusing with the splenocyte; and (c) instructions for use thereof.

The fused cells (hybridomas) or recombinant cells can be isolated using selective culture conditions or other suitable known methods, and cloned by limiting dilution or cell sorting, or other known methods. Cells that produce antibodies with the desired specificity can be selected by a suitable assay. The titer and the class of the antibody secreted into the culture supernatant can be evaluated by ELISA, by adding samples to the plates coated with the antigen, and detecting the signal using a labeled antibody against each class of human immunoglobulin.

The present invention also provides a method for producing an antibody, comprising the steps of: (a) culturing the hybridoma obtained by the above-described method, and (b) recovering a human antibody which binds to the antigen and that is produced by the hybridoma cell line.

In another embodiment, the subject invention provides a method of isolating a human antibody targeting an antigen of interest, the method comprising the steps of: (a) administering an antigen of interest to the reconstituted mouse capable of producing a fully human antibody as described herein; (b) obtaining a splenocyte that produces human antibodies specific for said antigen from said mouse; (c) fusing said splenocyte to a heteromyeloma to produce a hybridoma cell capable of producing a human antibody specific for said antigen; and (d) isolating a human antibody from said hybridoma cell that is specific for said antigen, thereby isolating a human antibody.

In another embodiment, the subject invention provides a humanized antibody isolated using a method comprising the steps of: (a) administering an antigen of interest to the reconstituted mouse capable of producing a fully human antibody as described herein; (b) obtaining a splenocyte that produces human antibodies specific for said antigen from said mouse; (c) fusing said splenocyte to a heteromyeloma to produce a hybridoma cell capable of producing a human antibody specific for said antigen; and (d) isolating a human antibody from said hybridoma cell that is specific for said antigen, thereby isolating a human antibody.

In another embodiment, the subject invention provides a method of isolating a human antibody specific for an antigen of interest, the method comprising the steps of: (a) administering an antigen of interest to a mouse capable of producing a fully human antibody; (b) obtaining a splenocyte that produces human antibodies specific for said antigen from said mouse; (c) fusing said splenocyte to the heteromyeloma as described herein to form a hybridoma cell capable of producing a human antibody specific for said antigen; and (d) isolating a human antibody from said hybridoma cell that is specific for said antigen, thereby isolating a human antibody.

In another embodiment, the subject invention provides a humanized antibody isolated according to the method comprising the steps of: (a) administering an antigen of interest to a mouse capable of producing a fully human antibody; (b) obtaining a splenocyte that produces human antibodies specific for said antigen from said mouse; (c) fusing said splenocyte to the heteromyeloma as described herein to form a hybridoma cell capable of producing a human antibody specific for said antigen; and (d) isolating a human antibody from said hybridoma cell that is specific for said antigen, thereby isolating a human antibody.

In one embodiment, adjuvants provided herein are used for generating antibodies of for use in therapy along with an antibody generated from the methods provided herein and such adjuvants include but are not limited to, complete freunds adjuvant or incomplete freunds adjuvant.

In one embodiment, the splenocytes used for fusing to an immortal cell line to form a hybridoma is a primed splenocyte. In another embodiment, the primed splenocytes is capable of producing antibodies specific for an antigen. One such antigen is exemplified herein and was used to immunize mice provided herein (CD44, see Example 1 below). In another embodiment, the splenocyte is a B-cell, a plasma cell or a B lymphocyte. In another embodiment, the splenocyte is a B-cell capable of producing an antigen-specific monoclonal human antibody.

In one embodiment, the heteromyeloma provided herein to which the splenocyte also provided herein is fused to, is arrived at by fusing a human immortal cell line with a murine immortal cell line. In another embodiment, the murine immortal cell line is a myeloma. In another embodiment, the human immortal cell line is a non-secreting human B-lymphocyte from a human lymphoma. In another embodiment, the heteromyeloma arrived at by the methods provided herein is also an immortal cell line. In another embodiment, the human immortal cell line used in generating the heteromyeloma is a human myeloma U-266, a lymph node cell from a patient with a B-cell lymphoma or a cell from a human nodular lymphoma. In another embodiment, murine immortal cell line used in generating the heteromyeloma is a Mouse myeloma X63-Ag8.653, murine Ag8 myeloma cell, or an NS-1 mouse myeloma cell line. NS-1 cells are cells used for the production of monoclonal antibodies by fusion with splenocytes. NS-1 cells are deficient in the gene for HGPRT (HPRT) and are killed in the presence of aminopterin. In hybridomas the lack of HGPRT in NS-1 cells is genetically complemented by the gene from mouse splenocytes, and as a consequence they survive selection in culture medium containing HAT.

In another embodiment, the subject invention provides a heteromyeloma cell produced by the fusion of the modified human lymphoma B cell line (OCI-LY-19, DSMZ no ACC 528) to the murine myeloma X63-Ag8.653 (ATCC CRL1580).

In another embodiment, the subject invention provides a method of producing a heteromyeloma cell comprising the step of fusing the modified human lymphoma B cell line (OCI-LY-19, DSMZ no ACC 528) to the murine myeloma X63-Ag8.653 (ATCC CRL1580), thereby producing a heteromyeloma.

In another embodiment, the subject invention provides a kit for preparing a heteromyeloma cell line, the kit comprising: (a) murine myeloma X63-Ag8.653 cells; (b) modified human B lymphoma OCI-LY-19, DSMZ no ACC 528 cells; and (c) instructions for use thereof.

In one embodiment, the heteromyeloma cell provided herein is produced by a method comprising the step of fusing the modified human lymphoma B cell line (OCI-LY-19, DSMZ no ACC 528) to the murine myeloma X63-Ag8.653 (ATCC CRL1580). In another embodiment, the fusion of the modified human lymphoma B cell line (OCI-LY-19, DSMZ no ACC 528) to the murine myeloma X63-Ag8.653 (ATCC CRL1580) takes place in the presence of poly(ethylene)glycol (PEG) 1540. In another embodiment, PEG serves to merge or fuse the two cells together into one cell. In another embodiment, fusion is carried out using the sendai virus. In yet another embodiment, fusion is carried out using any fusogenic reagent available in the art. It is to be understood that fusion of splenocytes with myelomas or heteromyelomas is well established in the art, for example, see (Oi & Herzenberg (1980) Selected Methods in Cellular Immunology, W. J. Freeman Co., San Francisco, Calif., p. 351) and that a skilled artisan when guided by the examples provided herein, would be able to carry out the necessary steps to arrive at a hybridroma provided herein.

In one embodiment, the heteromyeloma cell provided herein has a greater than 50% rate of fusion with a splenocyte.

In one embodiment, the heteromyeloma provided herein is K6H6/B5 (ATCC CRL1823) which is derived from a fusion of murine NS1-Ag4 myeloma cells with cells from a human nodular lymphoma (see Human antibodies for immunotherapy development generated via a human B cell hybridoma technology PNAS 2006 103:3557-3562). In another embodiment, the heteromyeloma provided herein is SHM-D33 which is derived from a fusion of human myeloma cell line FU-266, clone E-1(HAT sensitive, 8-azaguanine resistant and resistant to G-418) (x) murine myeloma P3X63Ag8.653. In one embodiment, the P3X63Ag8.653 cells are resistant to 8-azaguanine and are HAT sensitive. In another embodiment, the cells are used as fusion partners for producing hybridomas. In another embodiment, the cells do not secrete immunoglobulin. In another embodiment, the cells have been reported to be cholesterol auxotrophs due to a deficiency in 3-ketosteroid reductase activity.

In one embodiment, the non-secreting human lymphoma B cell line is maintained in culture and cloned in presence of G-418 and 8-azaguanine in order to obtain a G-418 and 8-azaguanine resistant and aminopterin sensitive clone. In another embodiment, the mouse myeloma is highly proliferative and does not secrete any light or heavy mouse immunoglobulin chains. In another embodiment, the mouse myeloma is 8-azaguanine and ouabaïn resistant but sensitive to G-418 and ouabaïn.

In one embodiment, the heteromyeloma has the following properties: it provides stable response to the selective medium; it fuses with the antigen-primed human lymphocytes (from humanized mice: GraftoMouse); it confers characteristics which optimize cloning procedures; it does not secrete immunoglobulin chains; it has a high proliferation rate; and it has a high number and a low segregation of human chromosomes in the parental cell line over a long period of time.

In one embodiment, a pharmaceutical composition containing the cord blood cells used for generating an antibody according to the methods of the present invention are, in another embodiment, administered to a non-human mammal, e.g. a mouse, by any method known to a person skilled in the art, such as parenterally, paracancerally, transmucosally, transdermally, intramuscularly, intravenously, intra-dermally, subcutaneously, intra-peritonealy, intra-ventricularly, intra-cranially, intra-vaginally or intra-tumorally.

In one embodiment, a pharmaceutical compositions containing the antibodies and compositions of the present invention are, in another embodiment, administered to a subject by any method known to a person skilled in the art, such as parenterally, paracancerally, transmucosally, transdermally, intramuscularly, intravenously, intra-dermally, subcutaneously, intra-peritonealy, intra-ventricularly, intra-cranially, intra-vaginally or intra-tumorally.

In one embodiment, provided herein is a method of treating a cancer or tumor further provided herein, the method comprising the step of administering to a subject an antibody provided herein. In another embodiment, provided herein is a method of preventing, inhibiting, or suppressing a cancer or a tumor growth, the method comprising the step of administering to a subject the antibody provided herein. In another embodiment, provided herein is a method for increasing remission in a subject previously affected by a cancer or tumor. In another embodiment, provided herein is a method for decreasing the incidence of a cancer or tumor, further provided herein, wherein the method comprises the step of administering to a subject the antibody obtained by the methods provided herein. Each possibility represents a separate embodiment of the invention.

In another embodiment, the subject invention provides a method of treating cancer, the method comprising the step of administering to a subject a humanized antibody isolated using a method comprising the steps of: (a) administering an antigen of interest to the reconstituted mouse capable of producing a fully human antibody as described herein; (b) obtaining a splenocyte that produces human antibodies specific for said antigen from said mouse; (c) fusing said splenocyte to a heteromyeloma to produce a hybridoma cell capable of producing a human antibody specific for said antigen; and (d) isolating a human antibody from said hybridoma cell that is specific for said antigen, thereby isolating a human antibody.

In another embodiment, the subject invention provides a method of preventing, inhibiting, or suppressing cancer, the method comprising the step of administering to a subject a humanized antibody isolated using a method comprising the steps of: (a) administering an antigen of interest to the reconstituted mouse capable of producing a fully human antibody as described herein; (b) obtaining a splenocyte that produces human antibodies specific for said antigen from said mouse; (c) fusing said splenocyte to a heteromyeloma to produce a hybridoma cell capable of producing a human antibody specific for said antigen; and (d) isolating a human antibody from said hybridoma cell that is specific for said antigen, thereby isolating a human antibody.

In another embodiment, the subject invention provides a method of treating cancer, the method comprising the step of administering to a subject a humanized antibody isolated according to the method comprising the steps of: (a) administering an antigen of interest to a mouse capable of producing a fully human antibody; (b) obtaining a splenocyte that produces human antibodies specific for said antigen from said mouse; (c) fusing said splenocyte to the heteromyeloma as described herein to form a hybridoma cell capable of producing a human antibody specific for said antigen; and (d) isolating a human antibody from said hybridoma cell that is specific for said antigen, thereby isolating a human antibody.

In another embodiment, the subject invention provides a method of preventing, inhibiting, or suppressing cancer, the method comprising the step of administering to a subject a humanized antibody isolated according to the method comprising the steps of: (a) administering an antigen of interest to a mouse capable of producing a fully human antibody; (b) obtaining a splenocyte that produces human antibodies specific for said antigen from said mouse; (c) fusing said splenocyte to the heteromyeloma as described herein to form a hybridoma cell capable of producing a human antibody specific for said antigen; and (d) isolating a human antibody from said hybridoma cell that is specific for said antigen, thereby isolating a human antibody.

In one embodiment, the antibody provided herein when used for treating a disease provided herein is also used in conjunction with a supplemental treatment regiment. For example, when treating cancer, such regiments include but are not limited to, surgery, chemotherapy, radiation, etc. In another embodiment, supplemental treatments can be carried out in a patient or administered to a patient prior to or after administering an antibody produced by the methods provided herein.

In one embodiment, the antigen provided herein is a viral antigen, a cancer-related antigen, or an allergy-related antigen.

In another embodiment, the viral antigen includes but is not limited to antigens from HIV, herpesvirus, parvovirus, HPV, and the like.

In another embodiment, the cancer-related antigen is a tumor antigen. Tumor antigens contemplated in the present invention include, but are not limited to, any of the various MAGEs (Melanoma-Associated Antigen E), including MAGE 1 (e.g., GenBank Accession No. M77481), MAGE 2 (e.g., GenBank Accession No. U03735), MAGE 3, MAGE 4, etc.; any of the various tyrosinases; mutant ras; mutant p53 (e.g., GenBank Accession No. X54156 and AA494311); and p97 melanoma antigen (e.g., GenBank Accession No. M12154). Other tumor-specific antigens include the Ras peptide and p53 peptide associated with advanced cancers, the HPV 16/18 and E6/E7 antigens associated with cervical cancers, MUC1-KLH antigen associated with breast carcinoma (e.g., GenBank Accession No. J03651), CEA (carcinoembryonic antigen) associated with colorectal cancer (e.g., GenBank Accession No. X98311), gp100 (e.g., GenBank Accession No. S73003) or MART1 antigens associated with melanoma, and the PSA antigen associated with prostate cancer (e.g., GenBank Accession No. X14810). The p53 gene sequence is known (See e.g., Harris et al. (1986) Mol. Cell. Biol., 6:4650-4656) and is deposited with GenBank under Accession No. M14694. Tumor antigens encompassed by the present invention further include, but are not limited to, Her-2/Neu (e.g. GenBank Accession Nos. M16789.1, M16790.1, M16791.1, M16792.1), NY-ESO-1 (e.g. GenBank Accession No. U87459), hTERT (aka telomerase) (GenBank Accession. Nos. NM003219 (variant 1), NM198255 (variant 2), NM 198253 (variant 3), and NM 198254 (variant 4), proteinase 3 (e.g. GenBank Accession Nos. M29142, M75154, M96839, X55668, NM 00277, M96628 and X56606) HPV E6 and E7 (e.g. GenBank Accession No. NC 001526) and WT-1 (e.g. GenBank Accession Nos. NM000378 (variant A), NMO24424 (variant B), NM 024425 (variant C), and NMO24426 (variant D)), cytochrome P450 1B1 (GenBank Accession Nos. NP_{—}000095), immature laminin receptor protein (OFA-iLRP) and human neutrophil elastase (GenBank Accession Nos. AAA36359.1). Thus, the present invention can be used as immunotherapeutics for cancers including, but not limited to, cervical, breast, colorectal, prostate, leukemia, lung cancers, and for melanomas. In another embodiment, the cancer includes but is not limited to, Hodgkin's lymphoma, non-Hodgkin's lymphoma, Burkett's lymphoma, chronic myeloid leukemia or any other lymphoma known in the art.

In one embodiment, the antibodies provided herein can be generated using various mouse cancer models, including, but not limited to, CD44 expressing acute myeloid leukemia models (AML), Her-2 expressing breast cancer models, PSA-expressing prostate tumor models and the like. It is to be understood that any such cancer or tumor model for the production of a human antibody for therapeutic and prophylactic use is encompassed by the methods provided herein. It is also to be understood that a skilled artisan would not limit himself/herself to the cancer/tumor models presented herein.

In another embodiment, the allergy-related antigen includes but is not limited to, soluble IgE.

In another embodiment, the invention also provides a human antibody produced by any of the methods of the invention. In another embodiment, the human antibody of the invention is an antibody belonging to the IgG class.

In one embodiment, the antibody produced by the methods provided herein is an anti-CD44 antibody. The CD44 antigen is a cell-surface glycoprotein involved in cell-cell interactions, cell adhesion and migration. In humans, the CD44 antigen is encoded by the CD44 gene. CD44 and its overexpression on the cell surface is known to be associated with certain malignancies including, but not limited to, leukemia and colon cancer.

In another embodiment, the antibody produced by the methods provided herein is an anti-CD133 antibody. CD133 is CD133 is a membrane molecule that has been associated with colorectal cancer.

In one embodiment, provided herein is a kit for obtaining a reconstituted immunodeficient mouse capable of producing a fully human antibody, the kit comprising: (a) neutralizing antibody specific for murine IL-2R beta; (b) human hematopoietic stem cells; (c) human TNF-α; and (d) instructions for use thereof.

Such a kit may contain other components, packaging, instructions, or other material to aid in obtaining a reconstituted immunodeficient mouse capable of producing a fully human antibody and aid in use of the kit.

In another embodiment, provided herein is a kit for preparing a hybridoma cell line capable of secreting a human antibody specific to an antigen of interest, the kit comprising: (a) a splenocyte producing human antibodies specific for said antigen from a reconstituted immunodeficient mouse administered a neutralizing antibody specific for murine IL-2R beta; human hematopoietic stem cells; human TNF-α; and the antigen of interest; (b) a heteromyeloma for fusing with the splenocyte; and (c) instructions for use thereof.

Also provided by the subject invention are kits for practicing the subject methods, as described above, specifically, provided herein are kits for preparing a hybridoma cell line capable of secreting a human antibody specific to an antigen of interest. The subject kits at least include one or more of: (a) a splenocyte that produces human antibodies specific for said antigen from a mouse capable of producing a fully human antibody; (b) a heteromyeloma cell produced by the fusion of the modified human lymphoma B cell line (OCI-LY-19, DSMZ no ACC 528) to the murine myeloma X63-Ag8.653 (ATCC CRL1580) for fusing with the splenocyte; and (c) instructions for use thereof. Also provided herein is a kit for preparing a heteromyeloma cell line, the kit comprising: (a) murine myeloma X63-Ag8.653 cells; (b) modified human B lymphoma OCI-LY-19, DSMZ no ACC 528 cells; and (c) instructions for use thereof. Other optional components of the kit include: buffers, instructions, etc., for obtaining an antibody or for performing an activity assay. The various components of the kit may be present in separate containers or certain compatible components may be precombined into a single container, as desired.

The subject kits typically further include instructions for using the components of the kit to practice the subject methods. The instructions for practicing the subject methods are generally recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or subpackaging) etc. In other embodiments, the instructions are present as an electronic storage data file present on a suitable computer readable storage medium, e.g. CD-ROM, diskette, etc. In yet other embodiments, the actual instructions are not present in the kit, but means for obtaining the instructions from a remote source, e.g. via the internet, are provided. An example of this embodiment is a kit that includes a web address where the instructions can be viewed and/or from which the instructions can be downloaded. As with the instructions, this means for obtaining the instructions is recorded on a suitable substrate.

In one embodiment, the term “subject” includes any human or nonhuman animal. The term “nonhuman animal” includes all vertebrates, e.g. mammals and non-mammals, such as nonhuman primates, sheep, dogs, cats, horses, cows, chickens, amphibians, reptiles, etc.

The practice of the invention employs, unless other otherwise indicated, conventional techniques or protein chemistry, molecular virology, microbiology, recombinant DNA technology, and pharmacology, which are within the skill of the art. Such techniques are explained fully in the literature. (See Ausubel et al., Current Protocols in Molecular Biology, Eds., John Wiley & Sons, Inc. New York, 1995; Remington's Pharmaceutical Sciences, 17th ed., Mack Publishing Co., Easton, Pa., 1985; and Sambrook et al., Molecular cloning: A laboratory manual 2nd edition, Cold Spring Harbor Laboratory Press—Cold Spring Harbor, N.Y., USA, 1989).

Unless defined otherwise, all technical and scientific terms used herein have the same meaning as is commonly understood by one of the skill in the art to which this invention belongs.

It is to be understood that all references mentioned herein are to be considered incorporated by reference in their entirety.

Having generally described this invention, a further understanding of characteristics and advantages of the invention can be obtained by reference to certain specific examples and figures which are provided herein for purposes of illustration only and are not intended to be limiting unless otherwise specified.

### EXAMPLES

### Materials and Methods

### Preparation and Culture of Umbilical Cord Blood (UCB) CD34+ Cells Mononuclear Cell Enrichment by Density Gradient Method

UCB sample was transferred into 50 mL conical tubes with 15 mL of cord blood in each tube and was diluted with 20 mL DPBS/EDTA. Samples were under-layered with 15 mL of Ficoll-Paque PLUS (GE Healthcare). Tubes were centrifuged at 400×g for 35 minutes at 18° C. without brake. Buffy coat interface layers were collected and diluted in DPBS/EDTA and washed three times by centrifugation at 18° C. with high brake. Centrifuge speed was 400×g for the first two wash steps and 300×g for the last wash step. Cells were centrifuged for 15 minutes for the first wash step and 10 minutes for the subsequent two washes. Washed cells in each tube were resuspended in 1 mL of DPBS/EDTA/BSA and pooled into one tube.

### CD34+ Cell Enrichment

Cells enriched for mononuclear cells (MNC) were incubated with a CD34+ antibody (Miltenyi Biotech) for 30 minutes at 6° C. as per manufacturer's protocol.

Unbound antibody was removed by washing the cells with PBS/EDTA/BSA.

Washed cells were processed on AutoMACS™ (Miltenyi Biotec) following manufacturer's instructions.

### Cell Culture and Expansion

Enriched CD34+ cells were centrifuged at 400×g for 10 minutes. Concentrated cells were resuspended in 1 mL of media consisting of equal volume of DPBS/EDTA/BSA and culture media.

Enriched cells were cultured for seven days in Stemspan (Stemcell Technologies) supplemented with a defined lipid cocktail (mixture of oleic acid, cholesterol and iron saturated transferrin (Sigma): 20 μl/ml, 50 μg/ml gentamycin (Lonza), 100 ng/ml SCF (Amgen), 100 ng/ml of Flt3-L (Amgen) and 100 ng/ml of Tpo (R& D Systems).

Immunization and Engraftment of Mice with Cultured UCB CD34+ Cells

Six-week-old female NOD/Scid mice were irradiated with 3.25 Gy (325 rads) from a ¹³⁷Cs source. A single i.p. injection of hTNF-α (R& D Systems, 50 μl, 0.5 μg per animal) and anti-mouse CD122 (0.5 mg) was given immediately after irradiation and 4 hours before CD34+ cells engraftment (5×10⁶ cells, i.v). Six weeks later, mice were immunized with CD44 antigen (50 μg of Fc human IgG-human CD44 fusion protein or 10⁷ blast cells from a leukemic AML1 patient) with Freund complete adjuvant. Four boosts with the same dose of antigen plus incomplete Freund adjuvant were performed every two weeks before fusion. Mice were injected daily i.p. with BLyS, 10 μg for four days before each boost.

### Human IgM and IgG ELISA

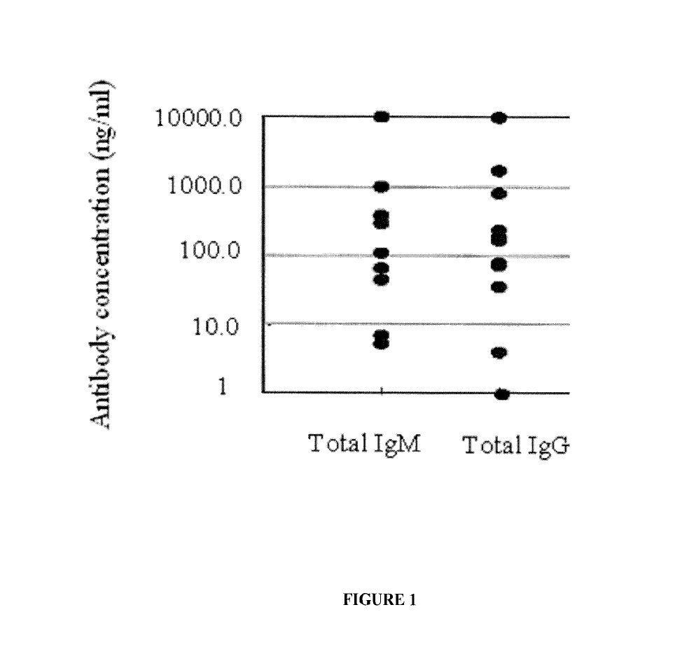

Levels of human IgM and IgG were measured in mice sera by an ELISA assay with anti-human IgG or anti-human IgM (Jackson) for coating and biotinylated mouse anti-human IgG1 for detection (FIG. 1).

FACS Analysis of Spleen from Humanized Mice

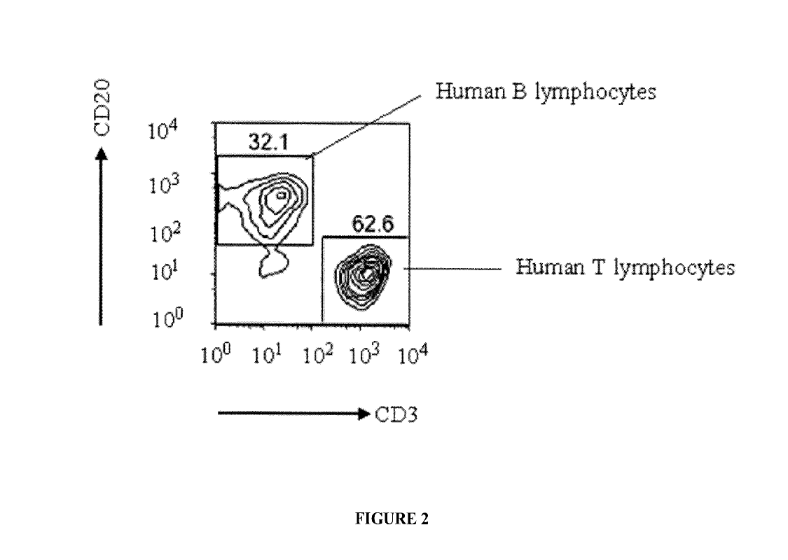

Spleens were digested with the rubber end of a plunger from a 2.5 ml syringe against the cell strainer to make single splenocytes. 0.5 ml 1×ACK lysing buffer (Invitrogen) was added to remove red blood cells. The cell suspension was placed on a cell strainer. To the collected cells were added 5 ml of FACS buffer. Cells were centrifuged at 1500 rpm, 10 min, 4° C. The supernatant was aspirated, and the cells were resuspended in 5-10 ml FACS buffer (PBS supplemented with 10% FCS and 0.1% sodium azide). After counting the cell number with a hemacytometer, the cells were diluted in FACS buffer (10⁸ cells/ml). For staining, 10 μl of the cell suspension were transferred to 96-well plate. An antibody mixture of anti-human CD20-PE (clone L27, BD Biosciences) and anti-human CD3-FITC (clone SK7, BD Biosciences) was added according to the manufacturer's instructions, and the reaction was incubated for 20 min at 4° C. 200 μl of FACS buffer were added to wash cells and the plate was centrifuged at 3000 rpm, 5 min, 4° C. The supernatant was aspired. This operation was repeated three times, and the cells were resuspended in 200 μl of FACS buffer and were ready for analysis by FACS to determine the presence of B cells and T cells. Samples were gated on live lymphocytes by forward and side scatter (FIG. 2).

Immunohistochemistry of Spleen from Humanized Mice

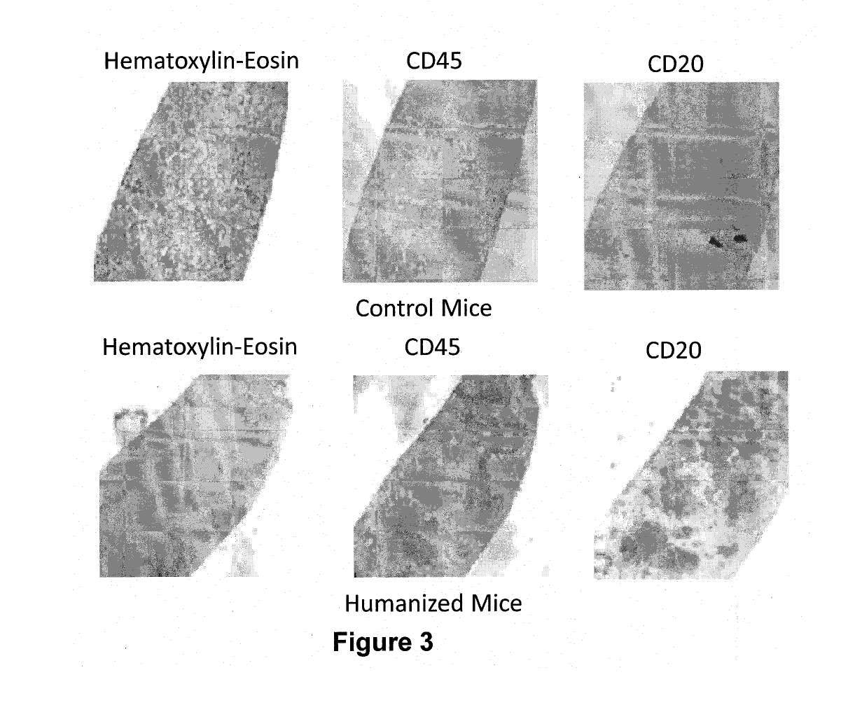

Fresh spleen dissected tissues (2-3 mm) were fixed for 48 h at room temperature in neutral buffered formalin, embedded in paraffin, and then 5 μm tissue sections were cut with a microtome. The sections were transferred onto Superfrost glass slides. Slides were allowed to dry overnight and were ready to use for immuno-histochemistry (IHC). After deparaffinization with 2 changes of xylene (5 min each), the slides were transferred to 2 changes of 100% alcohol (3 min each) and successive changes of 95%, 70% and 50% alcohol (3 min each). Endogeneous peroxidase activity was blocked by incubating sections in 3% H₂O₂ solution in methanol at room temperature for 10 min. The slides were rinsed twice for 5 min with PBS and incubated at 95° C., 10 min in 300 ml of citrate buffer, pH 6 for antigen retrieval. After 2 rinses with PBS, 100 μl of blocking buffer (10% FCS in PBS) were added to slides, 1 hour at room temperature. After draining off the blocking buffer, 100 μl of diluted (PBS with 0.5% BSA) anti-human CD20 Mab (clone L26, Abcam) or anti-human CD45 Mab (clone MEM-28, Abcam) were added to the slides for 1 hour in a humidified chamber. After 2 washes (5 min each) with PBS, 100 μl of diluted biotinylated goat ati-mouse IgG (Abcam) were applied to the slides for 1 hour at room temperature in a dark humidified chamber. After 2 washes (5 min each) with PBS, 100 μl of diluted streptavidin-HRP conjugate (Pierce) were applied to the slides for 30 min at room temperature in a dark humidified chamber. After 2 washes with PBS, staining was performed with 100 μl of DAB solution for 5 min. After 2 washes in PBS and rinsing with tap water for 15 min, the color of Mab staining was observed under a microscope. (FIG. 3).

### Heteromyeloma as Partner for Generating Fully Human Antibody Production

The heteromyeloma of the present invention is derived from fusing of the modified human lymphoma B cell line (OCI-LY-19, DSMZ no ACC 528) to the murine myeloma X63-Ag8.653 (ATCC CRL1580) using polyethylene glycol. The non secreting human lymphoma B cell line is maintained in culture and cloned in the presence of G-418 and 8-azaguanine in order to obtain a G-418 and 8-azaguanine-resistant and aminopterin-sensitive clone.

The mouse myeloma is highly proliferative and does not secrete any light and heavy mouse immunoglobulin chains. It is 8-azaguanine and ouabaïn-resistant but sensitive to G-418 and ouabaïn.

The main characteristics of the 2 partners are summarized in the table below:

TABLE 1

| Murine myeloma X63-Ag8.653 | Human B lymphoma cell line |
|---|---|
| G-418 sensitive | G-418 resistant |
| HAT sensitive | HAT sensitive |
| Ouabaïne resistant | Ouabaïne sensitive |
| 8-aza-guanine resistant | 8-aza-guanine resistant |

### Production of Fully Human Antibodies

NOD-SCID mice were injected (I.V.) with CD44 antigen in order to generate splenocytes that produce antibodies against these cells, in particular, the antigen CD44 expressed on the surface of these cells. CD44 is a type I transmembrane protein and functions as the major cellular adhesion molecule for hyaluronic acid, a component of the extracellular matrix. CD44 is expressed in most human cell types and is implicated in myeloid leukemia pathogenesis.

Once splenocytes (human B cells) are isolated from the mammal, the splenocytes are fused with immortalized heteromyeloma K6H6/B5 (which lack the hypoxanthine-guanine phosphoribosyltransferase [HGPRT] gene) cells (ATCC), using polyethylene glycol, according to the method of Kohler and Milstein (Nature. 1975 Aug. 7; 256(5517):495-7).

Fused cells are incubated in the HAT (Hypoxanthine Aminopetrin Thymidine) medium for 10 to 14 days. Aminopterin blocks the pathway that allows for nucleotide synthesis. Hence, unfused heteromyeloma cells die, as they cannot produce nucleotides by the de novo or salvage pathways because they lack HGPRT. Removal of the unfused heteromyeloma cells is necessary because they have the potential to outgrow other cells, especially weakly established hybridomas. Unfused B cells die as they have a short life span. In this way, only the B cell-myeloma hybrids survive, since the HGPRT gene coming from the B cells is functional. These cells produce antibodies (a property of B cells) and are immortal (a property of the heteromyeloma cells). The incubated medium is then diluted into multi-well plates to such an extent that each well contains only one cell. Since the antibodies in a well are produced by the same B cell, they will be directed towards the same epitope, and are thus monoclonal antibodies.

### Acute Myeloid Leukemia (AML1) Mouse Model

Mice were injected with AML1 cells at day 0 (I.V) and after allowing the tumor to grow, the same mice were administered a therapeutically effective dose of IMP11 or IgG control at day 10.

Survival studies, histologic studies, and FACS were carried out to determine the effectiveness of treatment with the IMP11 monoclonal antibody (see FIGS. 3-9).

### Example 1

### Preparation of Splenocytes and Heteromyelomas for Use in Generating Anti-CD44 (IMP11) Human Antibody-Producing Hybridoma Cells

Mice were injected with CD34+ cells and immunized with antigen (CD44), according to the materials and methods, and then the mice's immune response (including antibody production and adaptive immune response) was characterized in vitro.

Levels of IgM and IgG were measured in mouse sera (FIG. 1). Once it was confirmed that antibodies were being produced in the mice, spleens were harvested from the mice and stained with anti-human CD45, CD20, and CD3 to determine the presence of splenocytes (hematopoietic stem cells, B lymphocytes and T cells) using FACS (FIG. 2) and immunohistochemistry (FIG. 3). Samples were gated on live lymphocytes by forward and side scatter and on CD45 positive cells (FIG. 2).

To generate a heteromyeloma for using in generating a hybridoma, a somatic fusion between a myeloma and a human B lymphoma is carried out in the presence of polyethylene glycol (PEG 450), according to the methods of Kohler and Milstein, Nature. 1975 Aug. 7; 256(5517):495-7. Hybrid clones are selected in G-418 400 μg/ml and ouabaïne 0.5 μM, preserving their HAT sensitivity. The heteromyeloma shows the following properties: stable response to the selective medium, fuse with the antigen-primed human lymphocytes (from humanized mice: GraftoMouse), confer characteristics which optimize cloning procedures, does not secrete immunoglobulin chains, a high proliferation rate and a high number and a low segregation of human chromosomes in the parental cell line over a long period of time.

After generating heteromyelomas, splenocytes from antigen-primed NOD_SCID mice reconstituted with human B and T cells and heteromyeloma (K6H6/B5) were fused using PEG 450 in order to form an antibody-producing hybridoma. Splenocytes were transferred in a tube with 50 ml warm RPMI and centrifuged for 5 min at 300 g. At the same time, heteromyeloma cells (in 50 ml warm RPMI) were centrifuged to pellet. The supernatant was discarded. Heteromyeloma cells and splenocytes were mixed together at a ratio of 1 heteromyeloma: 4 splenocytes in 50 ml RPMI. The mixture was centrifuged (200 g, 5 min), and the supernatant discarded. One ml of 50% PEG 1500 was added by gently stirring over 90 sec at 37° C. 1 ml, 2 ml, 5 ml, and 10 ml of RPMI were added successively by gently stirring for 1 min each, and the tube was filled up with RPMI. The mixture was centrifuged and resuspended in 200 ml of selective medium (RPMI, penicillin 60 m/1, streptomycin 50 mg/1, glutamine 2 mM, sodium pyruvate 1 mM, 4 ml HAT 50×, FCS 10%, G-418 400 μg/ml and ouabaïne 0.5 μM) for hybridoma generation. The suspension was distributed in 10×96-well microplates (200 μl per well). Hybridoma growth was checked under an inverted microscope and medium was replaced by fresh selective medium every 6 days. After 15 days, the wells containing confluent hybridomas were screened for antibody secretion. Positive wells were expanded and rapidly cloned by limiting dilution.

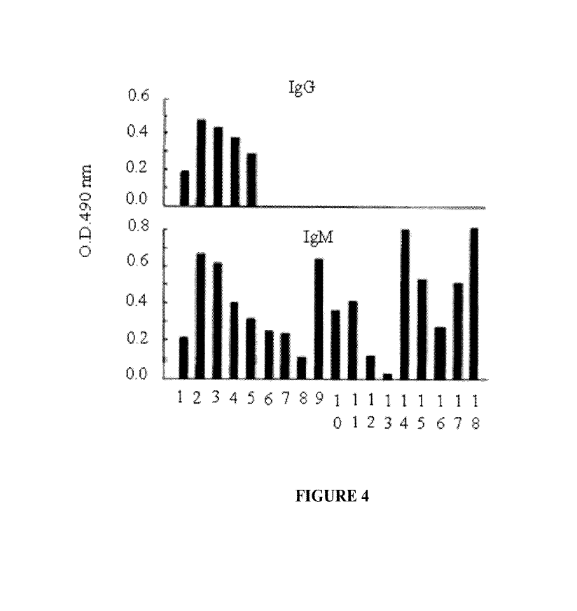

After cloning, stable positive clones (18 IgM, 5 IgG) producing specific antibodies against the antigen were obtained. ELISA tests were performed by coating the antigen (CD44) (FIG. 4).

After cloning of the antibody-producing hybridoma cells, stable positive clones (18 IgM, 5 IgG) producing specific antibodies against the antigen (CD44) were obtained. The selected clone is checked for rapid proliferation in normal culture medium and its stability.

Using the methods described above, an anti-CD44 monoclonal antibody IMP 111 was generated and tested in a human Acute Myeloid Leukemia (AML1) mouse model.

### Example 2

### Screening and Validating Antibody Functional Activity

The monoclonal antibodies generated in Example 1 are sequenced to determine whether the sequences are human sequences. ELISA tests were performed to screen for the antibody by coating the ELISA plates with the CD44 antigen (FIG. 4). Antibody function is further determined using the same process in order to determine antibody binding ability. Moreover, epitope mapping is carried out to analyze specificity of antibody binding.

Further, the affinity of binding of the monoclonal antibody to the target antigen is determined using an ELISA-based affinity assay.

### Example 3

### Kaplan Meier Survival Analysis after Treatment with Mab IMP11 (Anti-CD44) in Human AML1 Mouse Model

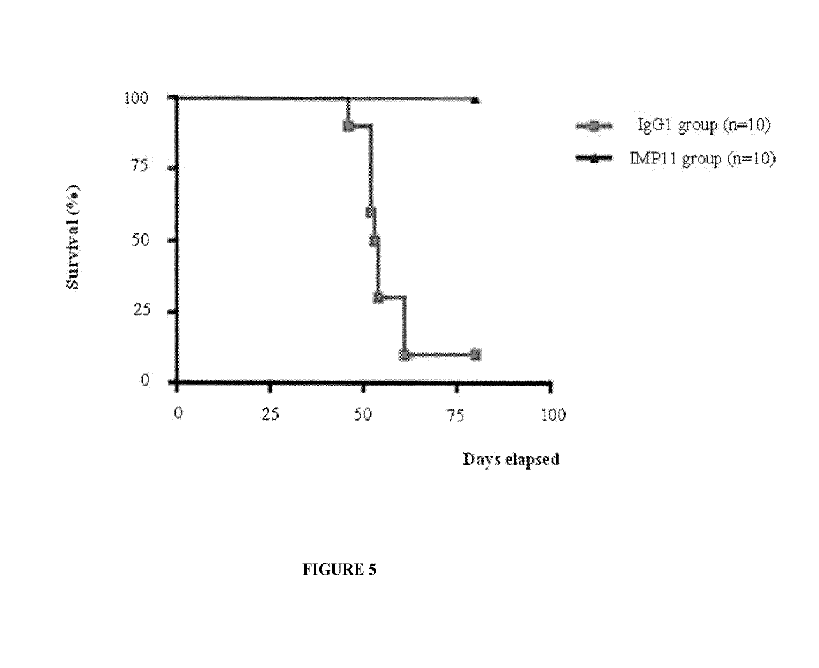

Human AML1 mouse models were treated with IMP 11 (anti-CD44) monoclonal antibodies (MAbs) (N=10) and IgG1 control (N=10), and a significant difference was found in percent survival between the IMP11 group and controls. There was a significant drop in percent survival in the IgG1 control group. By approximately day 47 post-treatment, survival began to significantly drop off, whereas in the IMP11-treated group, 100% survival of the mice persisted to a point beyond the experimentally determined length of time (until approximately 80 days), as opposed to the control group whose survival steadily declined until there was little to no survival by this same time period (FIG. 5).

### Example 4

### Induction of Antibody-Dependent Cell Phagocytosis (ADCP)

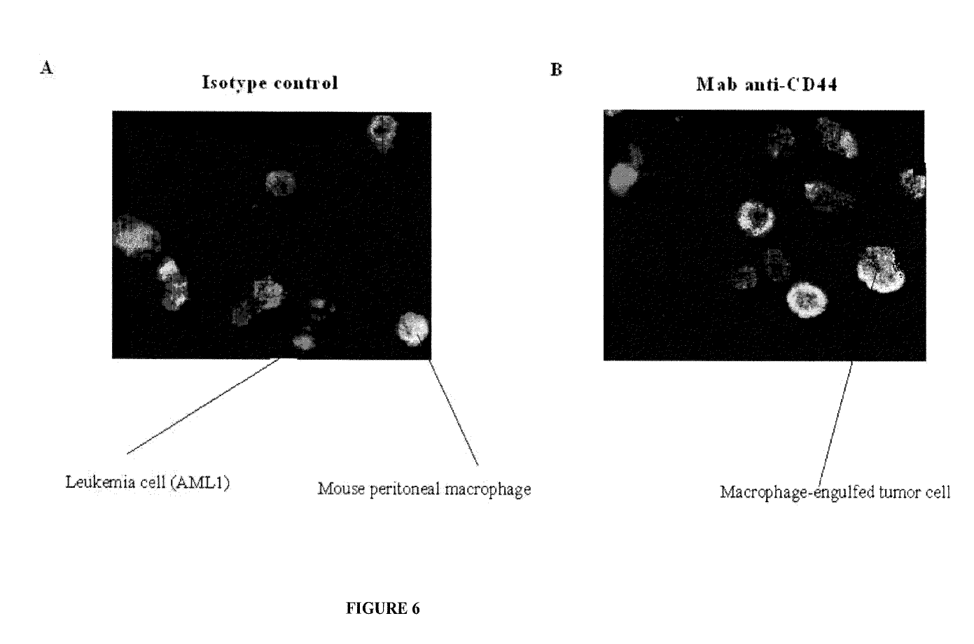

Mice treated with IMP11 MAbs had an increased incidence of ADCP of tumor cells, where tumor cells were phagocytosed by macrophages, as opposed to isotype control samples where no phagocytosis of tumor cells occurred (FIG. 6).

### Example 5

### Treatment of Human Acute Myeloid Leukemia Model Using IMP111 (Anti-CD44 Antibody)

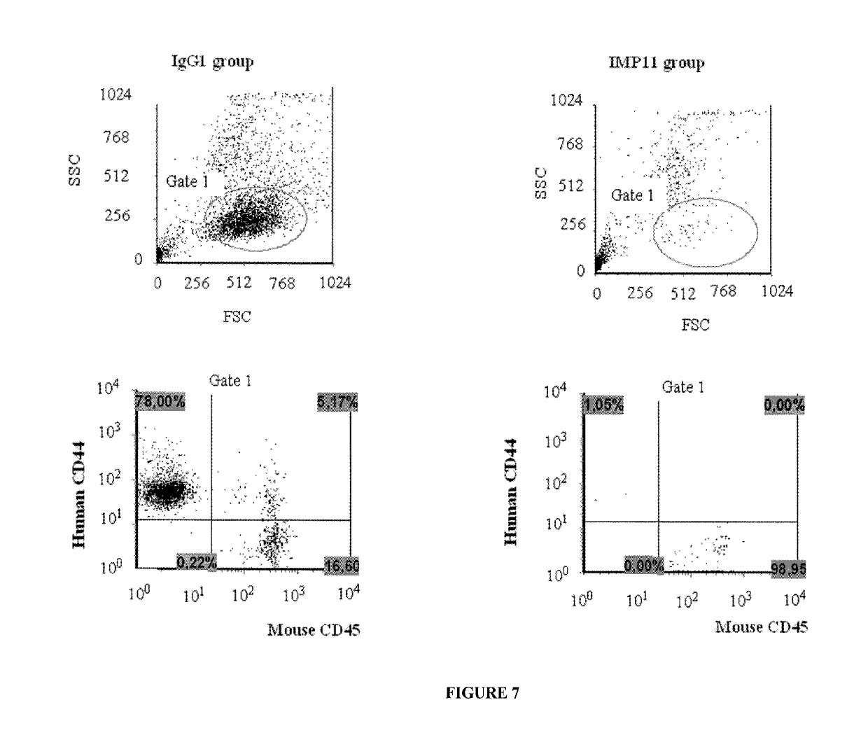

To determine the presence of human AML1 CD44+ cells in blood after treatment with IMP11, peripheral blood FACS analysis was carried out. By day 53 post-treatment, the number of human AML1 CD44+ cells had significantly declined, showing that treatment with IMP111 was effective in mediating elimination of these cells from the blood (FIG. 7). This was further evidenced by the clinical status of the treated group vs the control group, where in the former, the mice appeared healthy and in the latter, the mice were moribund (FIG. 7).

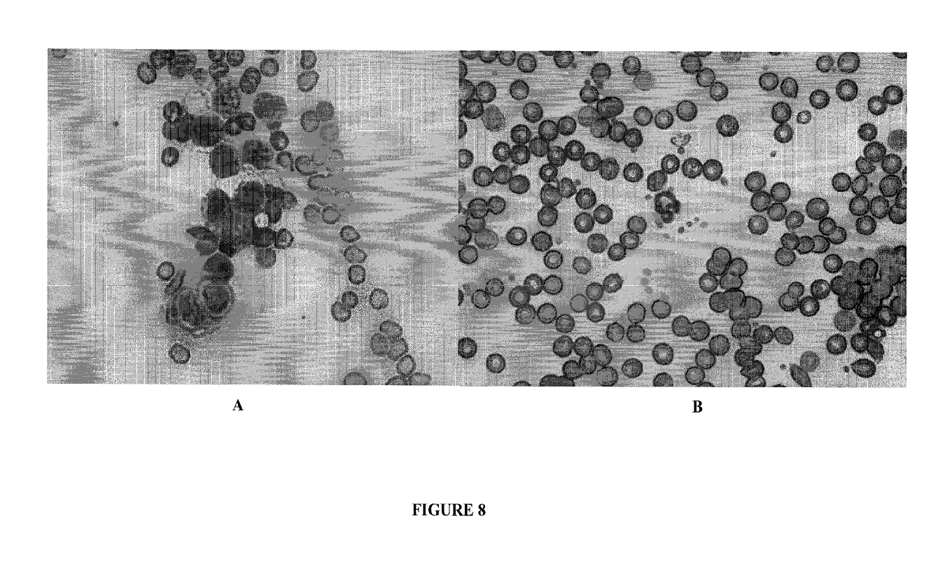

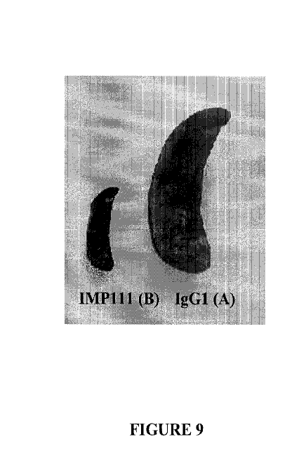

Moreover, blood smear (MGG) stains revealed that tumor cells were absent in IMP111 treated mice (FIG. 8B), as opposed to controls (FIG. 8B). Also, spleens were relatively larger in control groups as opposed to IMP111 treated groups, demonstrating a decreased activity in splenic function due to the absence of tumor cells (FIG. 9).

Having described preferred embodiments of the invention with reference to the accompanying drawings, it is to be understood that the invention is not limited to the precise embodiments, and that various changes and modifications may be effected therein by those skilled in the art without departing from the scope or spirit of the invention as defined in the appended claims.

1.-119. (canceled)

120. A method for obtaining a reconstituted mouse capable of producing a fully human antibody, the method comprising the steps of:
(a) administering a neutralizing antibody specific for murine IL-2R beta to an immunodeficient mouse;
(b) administering human hematopoietic stem cells to said mouse; and
(c) administering human TNF-α to the said mouse,
thereby obtaining a mouse capable of producing a fully human antibody.

121. A kit for obtaining a reconstituted immunodeficient mouse capable of producing a fully human antibody, the kit comprising:
(a) neutralizing antibody specific for murine IL-2R beta;
(b) human hematopoietic stem cells;
(c) human TNF-α; and
(d) instructions for use thereof.

122. An immunologically reconstituted mouse capable of producing a fully human antibody made according to the method of claim 1.

123. A method of isolating a human antibody targeting an antigen of interest, the method comprising the steps of:
(a) administering an antigen of interest to the immunologically reconstituted mouse capable of producing a fully human antibody of claim 3;
(b) obtaining a splenocyte that produces human antibodies specific for said antigen from said mouse;
(c) fusing said splenocyte to a heteromyeloma to produce a hybridoma cell capable of producing a human antibody specific for said antigen; and
(d) isolating a human antibody from said hybridoma cell that is specific for said antigen,
thereby isolating a human antibody.

124. A human antibody isolated according to the method of claim 4.

125. A method of treating cancer, the method comprising the step of administering to a subject the antibody of claim 5.

126. A method of preventing, inhibiting, or suppressing cancer, the method comprising the step of administering to a subject the antibody of claim 5.

127. A method of producing a hybridoma cell line capable of secreting a human antibody specific to an antigen of interest, the method comprising the steps of:
(a) administering an antigen of interest to the immunologically reconstituted mouse capable of producing a human antibody of claim 3;
(b) obtaining a splenocyte that produces human antibodies specific for said antigen from said mouse;
(c) fusing said splenocyte with a heteromyeloma to form a hybridoma cell capable of producing a human antibody specific for said antigen;
(d) identifying a hybridoma cell specific for said antigen; and
(e) expanding said hybridoma cell to produce a hybridoma cell culture capable of producing a human antibody specific for said antigen,
thereby producing a hybridoma cell line capable of secreting a human antibody specific to an antigen of interest.

128. A hybridoma cell line produced by the method of claim 8.

129. A kit for preparing a hybridoma cell line capable of secreting a human antibody specific to an antigen of interest, the kit comprising:
(a) a splenocyte producing human antibodies specific for said antigen from a reconstituted immunodeficient mouse administered a neutralizing antibody specific for murine IL-2R beta; human hematopoietic stem cells; human TNF-α; and said antigen of interest;
(b) a heteromyeloma for fusing with said splenocyte; and
(c) instructions for use thereof.

130. A heteromyeloma cell produced by the fusion of the modified human lymphoma B cell line (OCI-LY-19, DSMZ no ACC 528) to the murine myeloma X63-Ag8.653 (ATCC CRL1580).

131. A method of producing a heteromyeloma cell comprising the step of fusing the modified human lymphoma B cell line (OCI-LY-19, DSMZ no ACC 528) to the murine myeloma X63-Ag8.653 (ATCC CRL1580), thereby producing a heteromyeloma.

132. A kit for preparing a heteromyeloma cell line, the kit comprising:
(a) murine myeloma X63-Ag8.653 cells;
(b) modified human B lymphoma OCI-LY-19, DSMZ no ACC 528 cells; and
(c) instructions for use thereof.

133. A method of isolating a human antibody specific for an antigen of interest, the method comprising the steps of:
(a) administering an antigen of interest to a mouse capable of producing a fully human antibody;
(b) obtaining a splenocyte that produces human antibodies specific for said antigen from said mouse;
(c) fusing said splenocyte to the heteromyeloma of claim 11 to form a hybridoma cell capable of producing a human antibody specific for said antigen; and
(d) isolating a human antibody from said hybridoma cell that is specific for said antigen,
thereby isolating a human antibody.

134. A human antibody isolated according to the method of claim 14.

135. A method of treating cancer, the method comprising the step of administering to a subject the antibody of claim 15.

136. A method of preventing, inhibiting, or suppressing cancer, the method comprising the step of administering to a subject the antibody of claim 15.

137. A method of producing a hybridoma cell line capable of secreting a human antibody specific to an antigen of interest, the method comprising the steps of:
(a) administering an antigen of interest to a mouse capable of producing a fully human antibody;
(b) obtaining a splenocyte that produces human antibodies specific for said antigen from said mouse;
(c) fusing said splenocyte to the heteromyeloma of claim 11 to form a hybridoma capable of producing a human antibody specific for said antigen;
(d) identifying a hybridoma cell specific for said antigen; and
(e) expanding said hybridoma cell to produce a hybridoma cell culture capable of producing a human antibody specific for said antigen,
thereby producing a hybridoma cell line capable of secreting a human antibody specific to an antigen of interest.

138. A hybridoma produced by the method of claim 18.

139. A kit for preparing a hybridoma cell line capable of secreting a human antibody specific to an antigen of interest, the kit comprising:
(a) a splenocyte that produces human antibodies specific for said antigen from a mouse capable of producing a fully human antibody;
(b) a heteromyeloma cell produced by the fusion of the modified human lymphoma B cell line (OCI-LY-19, DSMZ no ACC 528) to the murine myeloma X63-Ag8.653 (ATCC CRL1580) for fusing with said splenocyte; and
(c) instructions for use thereof.
